(19) United States
(12) Patent Application Publication
(10) Pub. No.: US 2010/0305410 A1
(43) Pub. Date: Dec. 2, 2010

(54) APPLIANCE AND METHOD FOR CONTROLLING A PATIENT'S TONGUE DURING A MEDICAL PROCEDURE

(76) Inventor: Roland Young, Modesto, CA (US)

Correspondence Address: ITALIA IP, 3500 WEST OLIVE AVE., SUITE 300, BURBANK, CA, 91505, US

(21) Appl. No.: 12/848,992

(22) Filed: Aug. 2, 2010

(51) Int. Cl. A61B 13/00

(52) U.S. Cl. 600/240

(57) ABSTRACT

A medical appliance for positioning the tongue of a patient during a medical procedure includes a suction device having a hollow, bulbous body and an opening for contacting the patient's tongue. Depressing the body generates a suctioning at the opening, which joins the suction device to the tongue. A release valve may be attached to the suction device for releasing the suctioning force. The appliance also includes a retraction cord attached to the body of the suction device, which may be used to position or reposition the tongue to maintain an unobstructed airway. Optionally, one or more weighted clips may be attached to the retraction cord to further hold the tongue in a desired position. A method of controlling the tongue of a patient during a medical procedure using the appliance is also provided.

### TECHNICAL FIELD

The present disclosure is directed to the field of medical appliances and, more specifically, to an appliance and method for controlling the tongue of a non-intubated patient during a medical procedure.

### BACKGROUND

Many medical procedures relating to the mouth, such as dental surgery, do not require a patient to be intubated. Instead, the patient is allowed to breathe naturally. For this reason, it is necessary to maintain an open, unrestricted airway to avoid the risk of suffocation when the patient is anesthetized. Traditionally, the patient's tongue has been held with retractors, tongue forceps, or surgical instruments, which grab the tongue, but which must be held by the surgeon or his assistant.

What is needed in the industry is an appliance that safely positions the tongue of a patient during a medical procedure without the need for holding the appliance itself. Further, what is needed is an appliance that permits the tongue to be repositioned, if necessary, during the procedure without discomfort to the patient. In addition, a method for positioning the patient's tongue using such an appliance is also needed.

### SUMMARY

A medical appliance for positioning the tongue of a patient during a medical procedure is provided. The appliance includes a suction device having a hollow, bulbous body and an opening for contacting the patient's tongue. A suctioning force is generated at the opening when the body is depressed and the opening is in contact with the tongue. A release valve may be attached to the suction device for releasing the suctioning force. The appliance also includes a retraction cord attached to the body of the suction device, which may be used to position or reposition the tongue to maintain an unobstructed airway. Optionally, one or more weighted clips may be attached to the retraction cord to further hold the tongue in a desired position.

A method of controlling the tongue of a patient during a medical procedure is also provided. The method includes providing a medical appliance having a suction device, a release valve, and a retraction cord, as described above; positioning the opening of the suction body in contact with the patient's tongue; depressing the suction body to generate a suctioning force, thereby joining the suction device to the tongue; pulling the retraction cord to position the tongue to maintain an unobstructed airway.

### BRIEF DESCRIPTION OF THE DRAWING

The drawings, when considered in connection with the following description, are presented for the purpose of facilitating an understanding of the subject matter sought to be protected.

FIG. 1 is a schematic representation of a tongue-controlling appliance, as used within the mouth of a non-intubated patient.

### DETAILED DESCRIPTION

The present disclosure relates to a tongue-control appliance to aid in medical procedures relating to the mouth, such as oral surgery. Specifically, the appliance helps avoid the risk of suffocation associated with the patient's tongue retracting by helping to position the tongue in a stable position. The surgeon may use the appliance to conveniently and safely engage the patient's tongue and to position the tongue during surgery, such that an unobstructed airway is maintained.

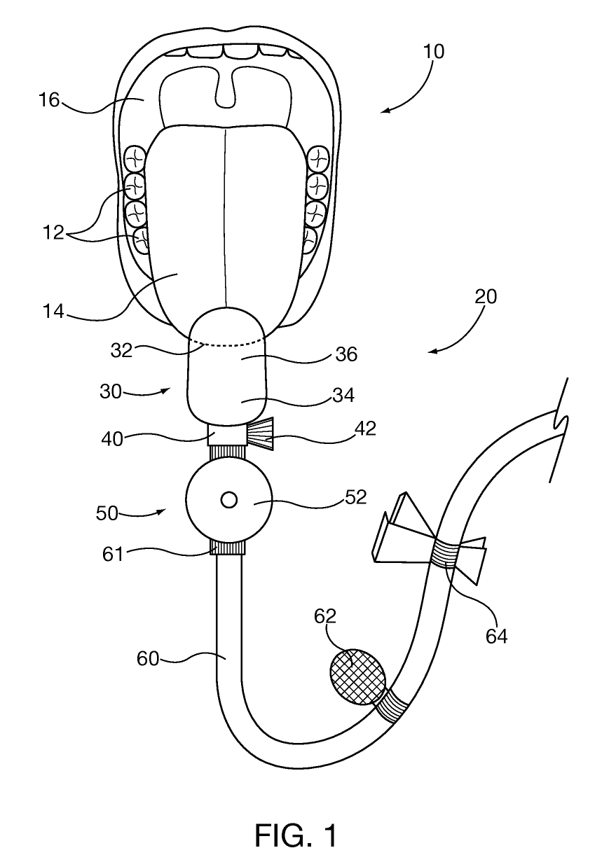

Turning now to FIG. 1, an appliance 20 is shown in contact with a patient's tongue 14. The patient's mouth 10 is open, and the tongue 14 is extended beyond the lower teeth 12. As shown, an unobstructed airway 16 is provided.

The appliance 20 includes a suction device 30, a release valve 40, and a retraction cord 60. The appliance 20 may further include a retraction cord connection assembly 50 and one or more weights 62 or clips 64 attached to the retraction cord 60.

The suction device 30 includes a bulbous, hollow body 36 having a closed end 34 and an opening 32, which engages the tongue 14. Depressing the closed end 34 of the hollow body 36 generates a suctioning force at the opening 32, such that, if the opening 32 is in contact with the tongue 16 when the suctioning force is created, the suctioning device 30 is joined to the tongue 14. The operation of the suction device 30 is easily accomplished and requires no special tools. Further, once the suction device 30 is joined to the tongue, the surgeon may use the retraction cord 60 and, optionally, the weights 62 and/or clips 64 to position or reposition the tongue during the medial procedure without the need for manually holding the tongue 16 in the desired position.

The release valve 40 may be attached to the closed end 34 of the body 36 of the suction device 30. For example, the release valve 40 and the bulbous body 36 may operate similarly to the inflation bulb of a blood pressure cuff. By turning a knob 42, the suctioning force is released, allowing the suction device 30 to be removed from the tongue 16. To reinstall the suctioning device 30, the knob 42 turned in the opposite direction to close the valve 40, so that the suctioning force may be regenerated.

Optionally, a retraction cord connection assembly 50 may be positioned between the release valve 40 and the retraction cord 60. The retraction cord assembly 50 may include a retraction wheel, or spool, 52, within which the retraction cord may be housed.

The retraction cord 60 is attached to the retraction cord assembly 50 or to the release valve 40, for example, by crimped connector 61. The retraction cord 60 may be a braided cord, a flat ribbon, or some other construction and may have a length of between about 12 inches and about 18 inches long.

One or more weighted clips 62 may be attached to the retraction cord 60, if desired, for greater control of the patient's tongue 14. The weighted clips 62 may each weigh between about 1 ounce and about 3 ounces. (NEED TO CONFIRM OR CHANGE) The weighted clips 62 may be removable and repositionable. Alternately, the weighted clips 62 may be slidable along the length of the retraction cord 60.

Instead of, or in addition to, the weighted clips 62, one or more clips 64 may be used to attach the retraction cord 60 to the shirt or smock of the patient. The clip 64 may hold the retraction cord 60 in a desired position that achieves the correct positioning of the tongue 14 to maintain an open airway 16. Again, the clips 64 may be removable and repositionable, or the clips 64 may be slidable along the retraction cord 60.

As may be appreciated by the preceding discussion, to control the tongue of a non-intubated patient during a medical procedure to maintain an unobstructed airway, the method includes: providing an appliance having a suction device 30, a release valve 40 attached to the suction device 30, and a retraction cord 60 attached to the release valve 40, the suction device 30 having a bulbous, hollow body 36 defining an opening 32 therethrough, such that the depression of the body 36 generates a suctioning force at the opening 32; positioning the opening 32 of the suction device 30 in contact with the patient's tongue 14; depressing the suction body 36 to generate a suctioning force, thereby joining the suction device 30 to the tongue 14; and pulling the retraction cord 60 to adjust the position of the tongue 14 to maintain an unobstructed airway 16.

During the surgical procedure, the surgeon (or his assistant) may use the retraction cord 60 to make periodic adjustments to the position of the patient's tongue 14, as needed. When the medical or surgical procedure and, optionally, the recovery period are completed, and when it is clear to the surgeon that the patient is able to adequately maintain his own airway, the appliance 20 is removed by opening the release valve 40 and expelling the suctioning force, thus disengaging the suction device 30 from the tongue 14. Unlike surgical techniques that use a suture stitch to control the tongue 14, the present appliance causes no damage or pain to the tongue 14. The appliance 20 may be disposed of after use.

Thus, the present appliance 20 represents a useful tool for a surgeon or doctor to control the position of a non-intubated patient's tongue 14 during a procedure.

What is claimed is:

1. An appliance for controlling the position of the tongue of a non-intubated patient during a medical procedure, the appliance comprising:
a suction device having a bulbous, hollow body defining an opening therethrough, the body being configured such that depression of the body generates a suctioning force at the opening;
a release valve attached to the body of the suction device for releasing the suction force; and
a retraction cord attached to the body of the suction device, the retraction cord being used to the control the position of the patient's tongue to maintain an unobstructed airway.

2. The appliance of claim 1, further comprising a weighted clip, the weighted clip being attached to the retraction cord for greater control of the position of the patient's tongue.

3. The appliance of claim 2, wherein multiple weighted clips are attached to the retraction cord.

4. The appliance of claim 2, wherein the weighted clip has a weight of between about 1 ounce and about 3 ounces. (THE CORRECT VALUES NEED TO BE SUBSTITUTED HERE.)

5. The appliance of claim 1, further comprising a clip attached to the retraction cord, the clip being configured to grasp a shirt or smock of the patient for greater control of the patient's tongue.

6. The appliance of claim 1, wherein the retraction cord is retractable and extends from a cord housing proximate the release valve.

7. The appliance of claim 1, wherein the retraction cord comprises a braided cord having a length of between about 12 inches and about 18 inches.

8. The appliance of claim 1, wherein the retraction cord comprises a flat ribbon.

9. The appliance of claim 1, wherein the suction device is disposable.

10. A method for controlling the tongue of a non-intubated patient during a medical procedure to maintain an unobstructed airway, the method comprising:
providing an appliance having a suction device, a release valve attached to the suction device, and a retraction cord attached to the release valve, the suction device having a bulbous, hollow body defining an opening therethrough, such that the depression of the body generates a suctioning force at the opening;
positioning the opening of the suction device in contact with the patient's tongue;
depressing the suction body to generate a suctioning force, thereby joining the suction device to the tongue; and
pulling the retraction cord to adjust the position of the tongue to maintain an unobstructed airway.

11. The method of claim 10, further comprising attaching a weighted clip to the retraction cord for greater control of the tongue.

12. The method of claim 11, further comprising attaching multiple weighted clips to the retraction cord.

13. The method of claim 10, further comprising attaching a clip to the retraction cord and grasping a shirt or smock of the patient with the clip for greater control of the tongue.

14. The method of claim 10, further comprising providing a retractable wheel for the retraction cord, such that the retraction cord may be spooled onto the wheel.

15. The method of claim 10, further comprising using the retraction cord to make periodic adjustments to the position of the tongue during the procedure.

16. The method of claim 10, further comprising opening the release valve to release suction force on the tongue.

17. The method of claim 10, further comprising disposing of the appliance after use.
